(19) United States
(12) Patent Application Publication
(10) Pub. No.: US 2007/0299396 A1
(43) Pub. Date: Dec. 27, 2007

(54) Pulsating irrigator apparatus for hygienic cleansing

(75) Inventor: Glen Rocklin, Culver City, CA (US)

Correspondence Address: Glen Rocklin, P.O. Box 4278, Culver City, CA, 90231-4278, US

(73) Assignee: Health Solution Medical Products Corporation, Culver City, CA (US)

(21) Appl. No.: 11/820,642

(22) Filed: Jun. 20, 2007

Related U.S. Application Data
(60) Provisional application No. 60/805,748, filed on Jun. 25, 2006.

(51) Int. Cl. A61M 37/00; A62B 7/00; A62B 9/00; A62B 18/00; A61M 16/00; A61M 15/00

(52) U.S. Cl. 604/131; 128/200.24

(57) ABSTRACT

A pulsatile irrigator apparatus is presented. The apparatus includes a solution tank with measurement scale for containing an aqueous solution, a piston driven pump assembly configured to generate an intermittent pulsing of the aqueous solution, an irrigation handle attachable to the pump assembly with a hose, an atomizer tip assembly attachable to the irrigation handle, an irrigator tip assembly attachable to the irrigation handle and attachment means for interchangeably attaching the atomizer tip and the irrigator tip to the irrigation handle where the apparatus provides a pulsating mist of the aqueous solution when the atomizer tip assembly is attached to the irrigation handle and the apparatus provides a pulsating flow of the aqueous solution when the irrigator tip is attached to the irrigation handle.

### CROSS-REFERENCE TO RELATED APPLICATIONS

The present Utility patent application claims priority benefit of the U.S. provisional application for patent Ser. No. 60/805,748 filed on Jun. 25, 2006 under 35 U.S.C. 119(e). The contents of this related provisional application are incorporated herein by reference for all purposes.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to irrigation of the sinuses and throat. More particularly, the invention relates to a pulsatile irrigator that provides a pulsating cleansing rinse and mist spray for sinus and throat cavities.

### BACKGROUND OF THE INVENTION

Irrigation devices may be used for tasks such as, but not limited to, cleaning wounds, and treating allergies and sinus problems. Furthermore, pulsatile irrigation is more effective than other methods such as, but not limited to, those reliant on gravity or bulbs and syringes. The following example is an illustration of the effectiveness of pulsatile irrigation.

Irrigation of wounds to remove bacteria and foreign material is an essential of wound management along with debridement. The effectiveness of saline lavage by pulsatile jet irrigation has been compared with conventional gravity flow and bulb syringe procedures. Experimental paravertebral incisional surface wounds in 234 randomized rats were either clean or traumatized and soiled. Wounds in 200 of the rats were seeded with E. coli (log 8.80). Swab specimens of each wound were taken at incision, after seeding, after irrigation, and at three, seven, and ten days after closure. Eulates of more than 1600 specimens were cultured. No anaerobes were found. Irrigation diminished bacterial counts in all wounds, however, only pulsatile jet irrigation brought about significant (e.g., without limitation, P less than 0:05) reduction of bacteria in each type of wound. After three days E. coli was significantly diminished in all wounds, regardless of irrigation or none, owing to host defense mechanisms. Nevertheless, clean contaminated wounds were infected at three days but not at seven days after lavage, while traumatized wounds remained infected at ten days except for those initially irrigated by pulsatile jet. Thus, pulsatile jet irrigation removed bacterial from experimental wounds more efficiently than conventional procedures.

A pulsating rinse also helps to restore ciliary function, promote drainage from the nose, and cleanse the nose of purulent material for the treatment of allergies and sinus ailments. The advantage of pulsatile irrigation is that in addition to active removal of thick mucus and contents, the rate of pulsation is such as to encourage the cilia to return to normal speed. The pulsation of a saline stream passing through the nose acts somewhat like a massage to encourage more circulation to the nasal membranes. Products such as biofilm, a major basic protein and resistant bacteria, may be removed as well. The leverage of intermittent pulses of solution provides greater leverage with less pressure. Furthermore, an intermittent pulsating throat rinse can also help prevent breath control problems, help soothe throat irritations, and cleanse debris and bacteria that accumulate and contribute to the formation of tonsil stones. No current devices are capable of producing both a pulsating cleansing rinse and mist spray atomizer function. There are pulsating irrigator units but none for sinus irrigation that can deliver both a fine mist pulsating mist spray (e.g., without limitation, 20-40 micron particle size) and a cleansing irrigation rinse (e.g., without limitation, 8-10 ml per second). Actual particle size will vary subject to pressure and flow rate. The primary benefit without regard to actual particle size is that of a pulsating atomized mist spray designed to moisturize and deliver medicated rinses.

Pulsatile irrigation or the delivery of intermittent pulses of water is far more effective at removing purulent material and bacteria than ordinary non-pulsating forms of irrigation. However, current methods of nasal irrigation rely on gravity, for example, without limitation, neti pots, or hand controlled pressure irrigation such as, but not limited to, squeeze bottles and bulb syringes. Other current irrigation methods are based on oral irrigators, for example, without limitation, the Water Pik™. However, these devices do not provide a mist spray function, which is particularly useful for the application of medicated rinses and moisturizers to the nose and throat.

In view of the foregoing, there is a need for an improved pulsatile irrigation device that can provide a cleansing rinse and a fine mist spray to the sinuses and the throat.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary pulsatile irrigation device, in accordance with an embodiment of the present invention;

FIGS. 2a and 2b illustrate an exemplary sinus mist tip of a pulsatile irrigation device, in accordance with an embodiment of the present invention. FIG. 2a is a cross-sectional view, and FIG. 2b is a side perspective view;

FIGS. 3a and 3b illustrate an exemplary throat mist tip of a pulsatile irrigation device, in accordance with an embodiment of the present invention. FIG. 3a is a cross-sectional view, and FIG. 3b is a side perspective view;

FIG. 4 is a side view of an exemplary pulsatile irrigator handle with an attached sinus irrigator tip, in accordance with an embodiment of the present invention; and

FIGS. 5a, 5b, 5c, and 5d illustrate an exemplary locking mechanism for a handle of an irrigation device, in accordance with an embodiment of the present invention. FIG. 5a is a side cross-sectional view of the locking mechanism in an locked position. FIG. 5b is a side cross-sectional view of the locking mechanism in a unlocked position. FIG. 5c is a top cross-sectional view of the locking mechanism in the locked position, and FIG. 5d is a top cross-sectional view of the locking mechanism in the unlocked position.

Unless otherwise indicated illustrations in the figures are not necessarily drawn to scale.

### SUMMARY OF THE INVENTION

To achieve the forgoing and other objects and in accordance with the purpose of the invention, a pulsating irrigator apparatus for hygienic cleansing is presented.

In one embodiment, a pulsatile irrigator apparatus is presented. The apparatus includes a solution tank for containing an aqueous solution, a piston driven pump assembly configured to generate an intermittent pulsing of the aqueous solution, an irrigation handle attachable to the pump assembly with a hose, an atomizer tip assembly attachable to the irrigation handle, an irrigator tip assembly attachable to the irrigation handle and attachment means for interchangeably attaching the atomizer tip and the irrigator tip to the irrigation handle where the apparatus provides a pulsating mist of the aqueous solution when the atomizer tip assembly is attached to the irrigation handle and the apparatus provides a pulsating flow of the aqueous solution when the irrigator tip is attached to the irrigation handle.

In another embodiment, a pulsatile irrigator apparatus is presented. The apparatus includes means for containing an aqueous solution, means for generating an intermittent pulsing of the aqueous solution, means for attaching to the pump assembly with a hose, means for providing a pulsating mist of the aqueous solution, means for providing a pulsating flow of the aqueous solution and means for interchangeably attaching the means for providing a pulsating mist and the means for providing a pulsating flow to the means for attaching to the pump assembly where the apparatus provides a pulsating mist of the aqueous solution and a pulsating flow of the aqueous solution.

In still another embodiment, a pulsatile irrigator apparatus is presented. The apparatus includes a housing containing a solution tank for containing an aqueous solution, a piston driven pump assembly configured to generate an intermittent pulsing of the aqueous solution, a switch for on and off control of the apparatus and a flow control to control the amount of solution that flows from the piston driven pump assembly. An irrigation handle is attachable to the pump assembly with a hose. The handle includes a push button for temporary shut off and pressure control of flow of the aqueous solution. An atomizer tip assembly is attachable to the irrigation handle. The assembly includes an atomizer bolt encased in an end of the assembly for atomizing the aqueous solution into a fine mist spray. An irrigator tip assembly is attachable to the irrigation handle. An attachment mechanism for provides for interchangeably attaching the atomizer tip and the irrigator tip to the irrigation handle where the apparatus provides a pulsating mist of the aqueous solution when the atomizer tip assembly is attached to the irrigation handle and the apparatus provides a pulsating flow of the aqueous solution when the irrigator tip is attached to the irrigation handle.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention will now be described in detail with reference to embodiments thereof as illustrated in the accompanying drawings.

As will be exemplified in some detail below, preferred embodiments of the present invention include advantageous aspects over conventional irrigation methods. For example, without limitation, pulsatile irrigation, or the delivery of intermittent pulses of water, is far more effective at removing purulent material and bacteria than ordinary non-pulsating forms of irrigation. The preferred embodiment of the present invention produces a calibrated pulsatile rinse of 1250 pulse per minute, effectively irrigating the nasal passages, lower sinuses, throat, and tongue.

The preferred embodiment produces both an intermittent cleansing pulse rinse for general cleansing and a pulsatile atomized spray mist to effectively deliver medicated or moisturizing rinses. Embodiments of the present invention also comprise a uniquely designed atomizer head and a specifically designed handle with a unique tip release locking system enabling the tip to create a fine pulsatile mist spray while generally ensuring that the tip is securely locked in place. Embodiments of the present invention also comprise a throat mist tip, which may be used with mouth rinses and other oral cleansing rinses, as well as a standard throat irrigator tip for general cleansing, useful for problems such as, but not limited to, sore throat irritation, tonsil stones, and cryptic tonsils.

Embodiments of the present invention may be used by a variety of users, for example, without limitation, those suffering from allergies and sinus problems, those suffering from bad breath, and those with tonsil stones or other throat infections or ailments. Physicians may also use embodiments of the present invention for the treatment of their patients.

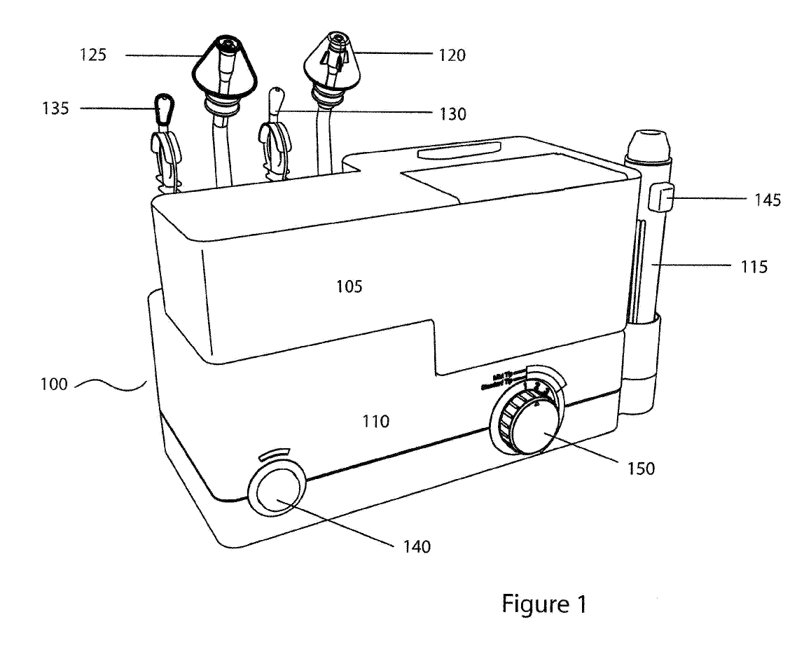

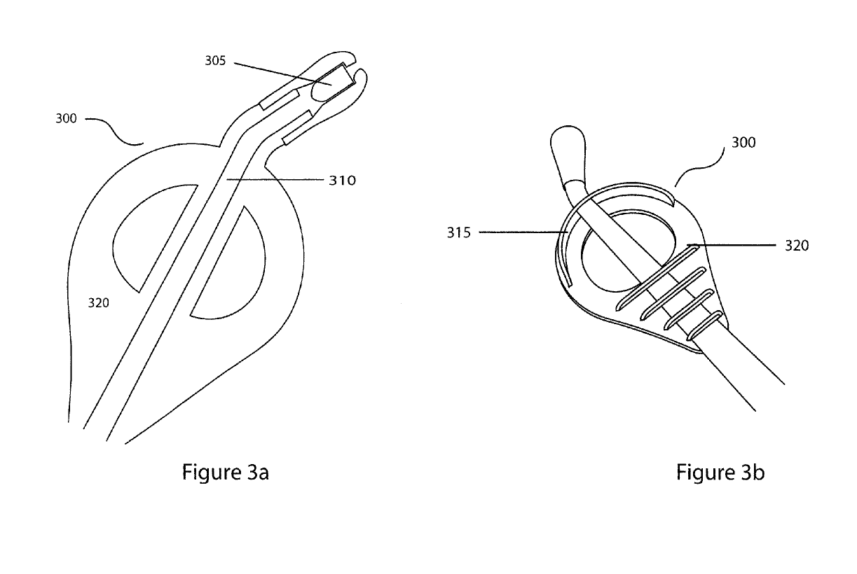

FIG. 1 illustrates an exemplary pulsatile irrigation device 100, in accordance with an embodiment of the present invention. In the present embodiment, irrigation device 100 comprises a high capacity water tank 105, a pulsatile irrigator 110, and a handle 115. The present embodiment comprises four attachments for handle 115, a sinus mist tip 120, a standard sinus irrigator tip 125, a throat mist tip 130, and a standard throat irrigator tip 135. Sinus mist tip 120 provides an atomized mist spray for the sinuses, sinus irrigator tip 125 provides a cleansing pulsatile rinse in the sinuses, throat mist tip 130 provides an atomized mist spray in the throat, and throat irrigator tip 135 provides a cleansing pulsatile rinse in the throat. Built into the design of throat mist tip 130 and throat irrigator tip 135 are two sided contoured tongue cleaners with four rows of ridges for an added oral hygiene benefit, as shown by way of example in FIGS. 3a and 3b.

In the present embodiment, irrigation device 100 features a push button micro switch 140 with LED display for easy on and off control, a touch push button 145 on handle 115 for temporary shut off and pressure control, and a step less water flow control 150 to control the amount of water that flows to handle 115. Water tank 105 comprises a measurement scale and hygienic flip lid.

Irrigation device 100 is designed to cleanse the nasal passages and lower sinuses with a gentle pulsating saline rinse. In the present embodiment, irrigation device 100 comprises a piston driven pump assembly designed to generate an intermittent pulsating rinse at 1250 pulses per minute (PPM). Alternate embodiments may pulse at various different speeds depending on the application of the irrigation device. Irrigation device 100 can provide both a general pulsating rinse and a pulsating moisturizing mist spray. In the present embodiment, the use of atomizer tips with 0.7 mm holes on sinus mist tip 120 and throat mist tip 125 enable the water flow to be atomized into a pulsatile mist spray function. The 0.7 mm hole creates a fine mist spray of 15 to 25 microns in particle size. The range of tolerance for the hole is ±0.03 mm. In alternate embodiments the hole of the atomizer tips may be larger or smaller to create a mist that is less fine or finer.

To provide the mist spray tips the preferred embodiment comprises both unique sinus and throat attachments, which generate a fine mist spray, and a connection and tip release mechanism that is able to contain the pressure created by the attachment so it is safe and effective.

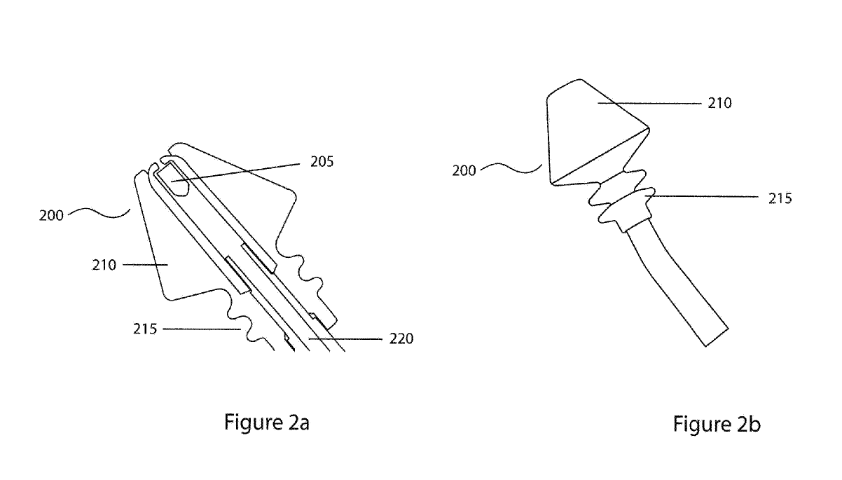

FIGS. 2a and 2b illustrate an exemplary sinus mist tip 200 of a pulsatile irrigation device, in accordance with an embodiment of the present invention. FIG. 2a is a cross-sectional view, and FIG. 2b is a side perspective view. The present embodiment comprises a pulsatile nasal/sinus atomizer mist spray function for both for general moisturizing, cleansing, and delivery of doctor recommended solutions and other rinses best served by a mist spray. An atomizer bolt 205 is encased in the end of sinus mist tip 200 that atomizes the water flow into a fine mist spray. In the present embodiment, the mist is 20 to 40 microns in particle size; however, this range may be broader in alternate embodiments depending on the particular application. In the present embodiment, atomizer bolt 205 has a 0.7 mm hole ±0.03 mm that enables the water flow to be atomized into a pulsatile mist spray function. In alternate embodiments, depending on the size of mist desired, the atomizer bolt may have a larger or smaller hole.

The present embodiment also comprises a silicone cosmetic bulb 210 to cover sinus mist tip 200. In alternate embodiments, the cosmetic bulb may be made of various other materials such as, but not limited to, other types of plastic or latex free materials. Yet other alternate embodiments may not include a cosmetic bulb. In the present embodiment, cosmetic bulb 210 has an accordion style base 215 that provides added flexibility for sinus mist tip 200 and comfort for the user. Furthermore, cosmetic bulb 210 may be removed from sinus mist tip 200, for example, without limitation, for cleaning or to replace with a new cosmetic bulb. Some embodiments may include different sizes of cosmetic bulbs for different users, for example, without limitation, an adult and a child size. In the present embodiment, the water travels through the handle and to atomizer bolt 205 through a water channel 220.

The sinus irrigator tip, shown by way of example in FIG. 1, is constructed in the same manner as sinus mist tip 200, except that atomizer bolt 205 is not included in the sinus irrigator tip. This enables a larger amount of water to pulse from the sinus irrigator tip to provide a general cleansing, pulsing rinse.

FIGS. 3a and 3b illustrate an exemplary throat mist tip 300 of a pulsatile irrigation device, in accordance with an embodiment of the present invention. FIG. 3a is a cross-sectional view, and FIG. 3b is a side perspective view. As with the sinus mist tip, shown by way of example in FIGS. 2a and 2b, the present embodiment comprises a pulsatile throat atomizer mist spray function for general moisturizing, cleansing, and delivery of doctor recommended solutions and other rinses best served by a mist spray. An atomizer bolt 305 is encased in the end of throat mist tip 300 that atomizes the water flow from water channel 310 into a fine mist spray. In the present embodiment, the mist has a size of 20-40 microns; however, various embodiments may use a broader range, depending on the application of the particular embodiment. In the present embodiment, atomizer bolt 305 has a 0.7 mm hole ±0.03 mm that enables the water flow to be atomized into a pulsatile mist spray function. In alternate embodiments, depending on the size of mist desired, the atomizer bolt may have a larger or smaller hole. The present embodiment also comprises a contoured tongue cleaner 315 and tongue depressor 320.

The throat irrigator tip, shown by way of example in FIG. 1, is constructed in the same manner as throat mist tip 300, except that atomizer bolt 305 is not included in the throat irrigator tip. This enables a larger amount of water to pulse from the throat irrigator tip to provide a general cleansing, pulsing rinse.

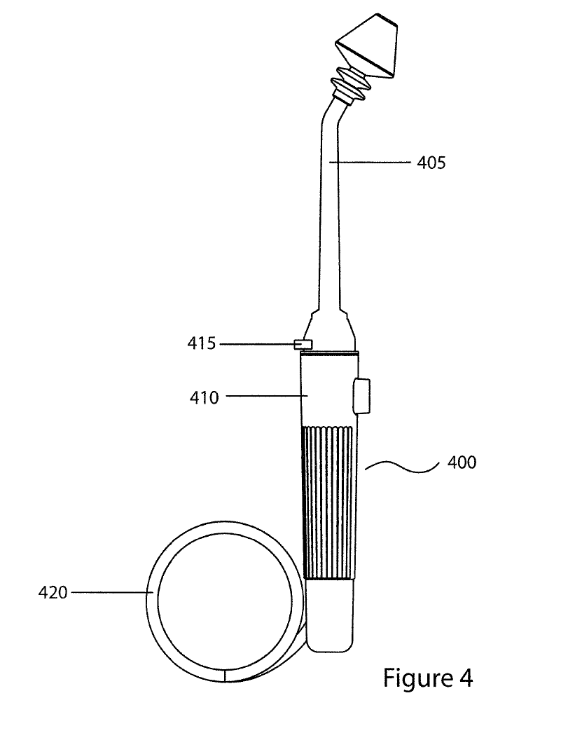

FIG. 4 is a side view of an exemplary pulsatile irrigator handle 400 with an attached sinus irrigator tip 405, in accordance with an embodiment of the present invention. In the present embodiment, the various tips connect and release from a base 410 of handle 400 by a locking mechanism. The locking mechanism has a release button 415 that releases the tip from base 410 when pressed. In the present embodiment, release button 415 is located on the back of base 410 near the top where the tips connect. In alternate embodiments the release button may be located in various other locations on the base of the handle. However, locating release button 415 on the back of base 410 in the present embodiment greatly reduces the risk of accidentally pressing release button 415 during use. The tips snap into base 410 without pressing release button 415, and the locking mechanism is a substantially abrasion free system. Those skilled in the art, in light of the present teachings, will recognize that the tips may connect to the base of the handle with various different means. For example, without limitation, the tips may screw into the base. In the present embodiment, handle 400 connects to the irrigation device with a hose 420.

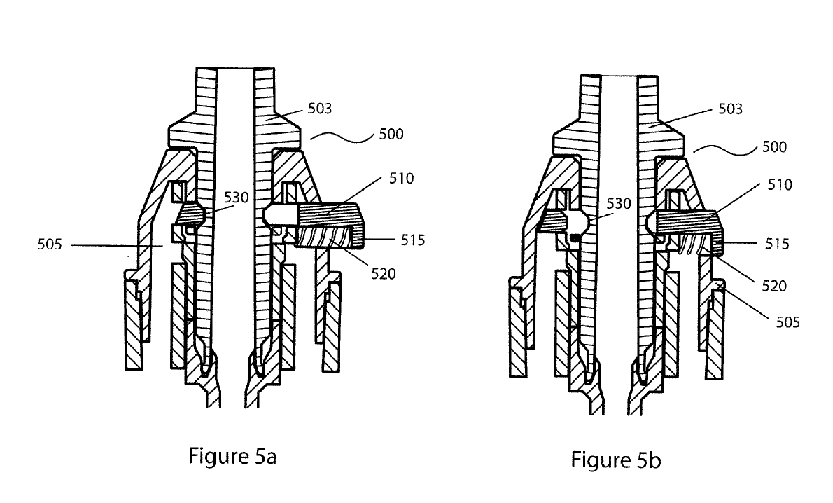

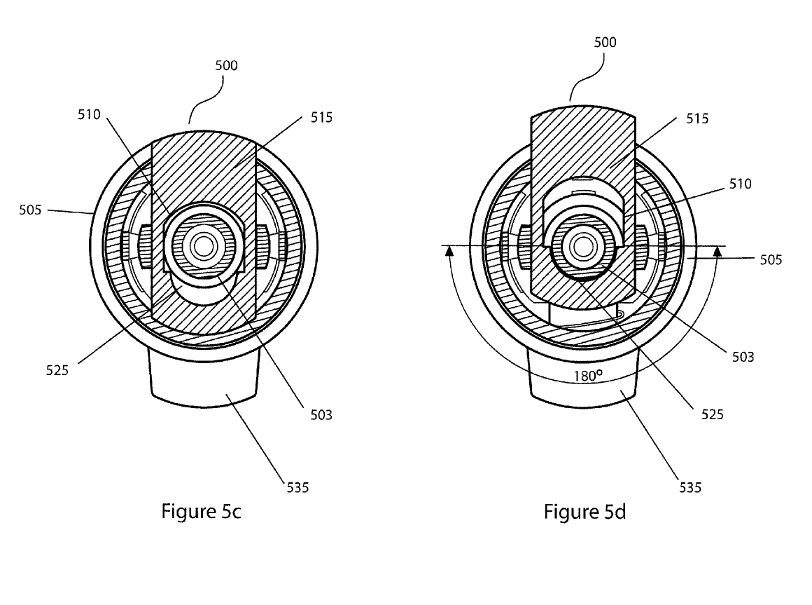

FIGS. 5a, 5b, 5c, and 5d illustrate an exemplary locking mechanism 500 for a handle 505 of an irrigation device, in accordance with an embodiment of the present invention. FIG. 5a is a side cross-sectional view of locking mechanism 500 in an locked position. FIG. 5b is a side cross-sectional view of locking mechanism 500 in a unlocked position. FIG. 5c is a top cross-sectional view of locking mechanism 500 in the locked position, and FIG. 5d is a top cross-sectional view of locking mechanism 500 in the unlocked position. In the present embodiment, locking mechanism 500 securely holds the mist spray tips in place while containing the pressure created by the atomizer function so the tip is not blown off by the pressure, making the irrigation device safe and effective. Locking mechanism 500 is a swivel collar lock mechanism, which enables an irrigator or mist tip 503 to turn freely while in place for easy use. In the present embodiment, locking mechanism 500 enables 180 degrees of contact around tip 503 in the locked position.

The base element of locking mechanism 500 is a locking slider portion 510 coupled with a release button 515. Locking mechanism 500 is self-locking, and a spring 520 with minimal spring force holds locking slider portion 510 in the locked position. When release button 510 is pressed, locking slider portion 510 slides into the unlocked position and tip 503 may be removed. In the locked position an indentation 525 of locking slider portion 510 is engaged into a notch 530 in tip 503. In the unlocked position, when release button 515 is pressed, indentation 525 is removed from notch 530 so that tip 503 may be pulled out of base 505. In the present embodiment, locking mechanism 500 allows smooth inserting of tip 503 without activating release button 515. Also, locking mechanism 500 is a substantially abrasion free system. Also shown in FIGS. 5c and 5d is a touch push button 535 for temporary shut off and pressure control of the irrigation device.

In typical use of an irrigation device in accordance with embodiments of the present invention, a user fills the tank of the irrigation device with water, saline (e.g., without limitation, approximately 16 oz.), or other solution such as, but not limited to, medicated rinses and moisturizers, and uses either a cleansing irrigator tip for the sinuses or throat or a moisturizing mist spray tip for the sinuses or throat. Once turned on, the irrigation device produces a gentle pulsing rinse or mist spray to cleanse the nasal passages, lower sinuses, throat, or tongue. The preferred embodiment is designed to deliver an intermittent pulse of 1200-1250 PPM. The pulsating rinse helps to restore ciliary function, promote drainage from the nose, and cleanse the nose of purulent material. When used with the throat irrigator tip, the irrigation device delivers a refreshing pulsating rinse for the throat and tongue for breath control problems, to help sooth sore throat irritation, and for use with tonsil stones. Some embodiments may also include packets of powdered saline so that the user can fill the tank with saline.

A self coiling, flexible, plastic hose connects the piston driven pump assembly within the irrigator to the handle delivering an intermittent pulsing aqueous solution to the respective cleansing or spray mist tips (sinus and throat).

Having fully described at least one embodiment of the present invention, other equivalent or alternative means for implementing a pulsatile irrigation device according to the present invention will be apparent to those skilled in the art. For example, without limitation, the above embodiments were described as delivering a rinse or mist of water; however, these embodiments may use various other solutions for the rinse or mist such as, but not limited to, medicated solutions, saline, moisturizers, etc. The invention has been described above by way of illustration, and the specific embodiments disclosed are not intended to limit the invention to the particular forms disclosed. The invention is thus to cover all modifications, equivalents, and alternatives falling within the spirit and scope of the following claims.

What is claimed is:

1. A pulsatile irrigator apparatus comprising:
a solution tank with measurement scale for containing an aqueous solution;
a piston driven pump assembly configured to generate an intermittent pulsing of said aqueous solution;
an irrigation handle attachable to said pump assembly with a hose;
an atomizer tip assembly attachable to said irrigation handle;
an irrigator tip assembly attachable to said irrigation handle; and
attachment means for interchangeably attaching said atomizer tip and said irrigator tip to said irrigation handle where the apparatus provides a pulsating mist of said aqueous solution when said atomizer tip assembly is attached to said irrigation handle and the apparatus provides a pulsating flow of said aqueous solution when said irrigator tip is attached to said irrigation handle.

2. The apparatus as recited in claim 1, in which said atomizer tip assembly comprises an atomizer bolt encased in an end of said assembly for atomizing said aqueous solution into a fine mist spray.

3. The apparatus as recited in claim 1, in which said irrigation handle comprises a push button for temporary shut off and pressure control of flow of said aqueous solution.

4. The apparatus as recited in claim 1, in which said atomizer tip assembly and said irrigator assembly tip are configured for use on a person's nasal passages and sinus cavities.

5. The apparatus as recited in claim 4, further comprising:
a throat atomizer tip assembly attachable by said attachment means for providing a pulsating mist of said aqueous solution to a person's throat; and
a throat irrigator tip assembly attachable by said attachment means for providing a pulsating flow of said aqueous solution to a person's throat.

6. The apparatus as recited in claim 5, in which said throat atomizer tip assembly comprises an atomizer bolt encased in an end of said assembly for atomizing said aqueous solution into a fine mist spray.

7. The apparatus as recited in claim 4, in which said atomizer tip assembly and said irrigator tip assembly comprise removable cosmetic bulbs covering at least an end of said assemblies where said bulbs are configured for comfort of a person using the apparatus.

8. The apparatus as recited in claim 5, in which said throat atomizer tip assembly and said throat irrigator tip assembly comprise contoured tongue cleaners and tongue depressors.

9. The apparatus as recited in claim 1, in which said solution tank and said piston driven pump assembly are contained in a housing.

10. The apparatus as recited in claim 9, in which said housing comprises a switch for on and off control of the apparatus.

11. The apparatus as recited in claim 10, in which said housing comprises a flow control to control the amount of solution that flows to said irrigation handle.

12. The apparatus as recited in claim 11, in which said housing comprises means for retaining said irrigation handle, atomizer tip assembly, irrigator tip assembly, throat atomizer tip assembly, and throat irrigator tip assembly.

13. A pulsatile irrigator apparatus comprising:
means for containing an aqueous solution;
means for generating an intermittent pulsing of said aqueous solution;
means for attaching to said means for generating an intermittent pulsing with a hose;
means for providing a pulsating mist of said aqueous solution;
means for providing a pulsating flow of said aqueous solution; and
means for interchangeably attaching said means for providing a pulsating mist and said means for providing a pulsating flow to said means for attaching to said pump assembly where the apparatus provides a pulsating mist of said aqueous solution and a pulsating flow of said aqueous solution.

14. The apparatus as recited in claim 13, further comprising means for housing said means for containing an aqueous solution and means for generating an intermittent pulsing of said aqueous solution.

15. A pulsatile irrigator apparatus comprising:
a housing comprising:
a solution tank with measurement scale for containing an aqueous solution;
a piston driven pump assembly configured to generate an intermittent pulsing of said aqueous solution;
a switch for on and off control of the apparatus; and
a flow control to control the amount of solution that flows from said piston driven pump assembly;
an irrigation handle attachable to said pump assembly with a hose, said handle comprising a push button for temporary shut off and pressure control of flow of said aqueous solution;
an atomizer tip assembly attachable to said irrigation handle, said assembly comprising an atomizer bolt encased in an end of said assembly for atomizing said aqueous solution into a fine mist spray;
an irrigator tip assembly attachable to said irrigation handle; and
attachment mechanism for interchangeably attaching said atomizer tip and said irrigator tip to said irrigation handle where the apparatus provides a pulsating mist of said aqueous solution when said atomizer tip assembly is attached to said irrigation handle and the apparatus provides a pulsating flow of said aqueous solution when said irrigator tip is attached to said irrigation handle.

16. The apparatus as recited in claim 15, in which said attachment mechanism comprises a swivel collar lock mechanism.

17. The apparatus as recited in claim 16, in which said swivel collar lock mechanism comprises a locking slider portion coupled with a release button.

18. The apparatus as recited in claim 17, in which said swivel collar lock mechanism is self-locking, and a spring holds said locking slider portion in a locked position.

19. The apparatus as recited in claim 18, in which when said release button is pressed, said locking slider portion slides into an unlocked position.

20. The apparatus as recited in claim 19, in which swivel collar locking mechanism allows smooth insertion of said atomizer and irrigation tip assemblies without activating said release button, and said swivel collar locking mechanism is a substantially abrasion free system.
